SEQUENCE LISTING (6 sequences)

SEQ ID NO: 1 - DNA, 20 bp, Artificial Sequence
  Description of Artificial SequenceSynthetic DNA (primer)
cctaccaggacaaggtctcg

SEQ ID NO: 2 - DNA, 24 bp, Artificial Sequence
  Description of Artificial SequenceSynthetic DNA (primer)
ccatcattgacattgcacccatgg

SEQ ID NO: 3 - DNA, 20 bp, Artificial Sequence
  Description of Artificial SequenceSynthetic DNA (primer)
cctaccaggacaaggtctcg

SEQ ID NO: 4 - DNA, 20 bp, Artificial Sequence
  Description of Artificial SequenceSynthetic DNA (primer)
gtagcctcgctgtcctcaag

SEQ ID NO: 5 - DNA, 24 bp, Artificial Sequence
  Description of Artificial SequenceSynthetic DNA (primer)
ccatcattgacattgcacccatgg

SEQ ID NO: 6 - DNA, 23 bp, Artificial Sequence
  Description of Artificial SequenceSynthetic DNA (primer)
gttagtttcctgtctctgccttg

(19) United States
(12) Patent Application Publication
(10) Pub. No.: US 2007/0116676 A1
(43) Pub. Date: May 24, 2007

(54) Method for three-dimensional cartilage tissue engineering using bone marrow cells in tissue engineering bone marrow cells in simulated microgravity environment

(76) Inventors: Naoka Kida, Ibaraki (JP); Toshimasa Uemura, Ibaraki (JP); Junzo Tanaka, Ibaraki (JP); Yoshimi Oyabu, Ibaraki (JP)

Correspondence Address: SUGHRUE MION, PLLC, 2100 PENNSYLVANIA AVENUE, N.W., SUITE 800, WASHINGTON, DC, 20037, US

(21) Appl. No.: 10/581,911

(22) PCT Filed: Dec. 2, 2004
(86) PCT No.: PCT/JP04/18339

§ 371 Date: Jun. 7, 2006

(30) Foreign Application Priority Data
Dec. 11, 2003 (JP) 2003-413758
Mar. 29, 2004 (JP) 2004-096686

(51) Int. Cl. A61K 35/14

(52) U.S. Cl. 424/93.7; 435/372

(57) ABSTRACT

This invention provides a method for three-dimensional cartilage tissue engineering by culturing bone marrow cells in a simulated microgravity environment that is realized by a bioreactor such as an RWV.

### TECHNICAL FIELD

The present invention relates to a method for three-dimensional cartilage issue engineering using bone marrow cells in a simulated microgravity environment.

### BACKGROUND ART

In recent years, techniques for repairing cartilage defects have been actively studied in the orthopedics field and some such techniques have been put to practical use. Specifically, cartilage cells isolated from the autologous cartilage of a patient are cultured and grown in vitro and then transplanted into defects. When cartilage cells are two-dimensionally cultured in a vessel such as a petri dish, however, they are dedifferentiated and converted into fibroblasts. This results in a loss of the original phenotype of cartilage cells, such as the capacity for cartilaginous matrix formation, and transplantation of such cells cannot give satisfactory therapeutic effects.

Three-dimensional culture can overcome such drawbacks; however, cells having a specific gravity that is somewhat higher than that of water sink in a culture medium on the ground, where everything is continuously affected by gravity, resulting in a two-dimensional culture. In general, therefore, an adequate scaffold is necessary to be used for completing three-dimensional culture.

Meanwhile, three-dimensional tissue engineering has been attempted using stirred fermentor. With such conventional techniques, however, considerable mechanical stimuli and damages are imposed on cells. Accordingly, it is difficult to obtain a large tissue mass. Even if a large tissue mass were to be obtained, the inner region of the formed tissue is likely to become necrotic.

In order to overcome such drawbacks, there are sets of bioreactors designed to optimize gravity. For example, the Rotating Wall Vessel (RWV) bioreactor is a NASA-developed rotary bioreactor equipped with gas exchange means (for example, U.S. Pat. No. 5,002,890). The RWV bioreactor, which is a horizontal cylindrical bioreactor, is filled with a culture medium, the cells are sowed therein, and the bioreactor rotates along the horizontal axis of the cylinder to culture cells. Because of the stress resulting from rotation, a microgravity environment is realized in the bioreactor, which provides gravity that is approximately 1/100 of the ground gravity. Accordingly, cells can grow while being homogeneously suspended in a culture medium, and they aggregate to form a large tissue mass.

In addition to the RWV bioreactor, several types of apparatuses that realize a simulated microgravity environment, such as the Rotary Cell Culture System™(RCCS) (Synthecon Incorporated) and a 3D-clinostat, have been developed (for example, JP Patent Publication (Unexamined) Nos. 8-173143 (1996), 9-37767 (1997), and 2002-45173), and they have been put to practical use. Further, results of cell culture in such a simulated microgravity environment have been already published as patents or articles (for example, U.S. Pat. Nos. 5,153,133, 5,155,034, 6,117,674, and 6,416,774). Regarding cartilage tissue engineering in a simulated microgravity environment, a method whereby a composite of a scaffold such as PLGA and cartilage cells is prepared to engineer cartilage tissue is known.

Extraction of autologous cartilage for cartilage tissue regeneration therapy involves a considerable damage imposed on healthy tissue, and the amount of extraction is disadvantageously limited. Accordingly, a technique of effective cartilage tissue regeneration in vitro, which involves the use of cells other than cartilage cells, has been awaited.

### DISCLOSURE OF THE INVENTION

The present invention provides a technique for three-dimensionally engineered cartilage tissue without damaging autologous cartilage.

The present inventors have conducted concentrated studies in order to overcome the drawbacks of conventional techniques. As a result, they conceived of the use of mesenchymal stem cells contained in bone marrow instead of autologous cartilage and differentiation and proliferation thereof to result in cartilage cells. With such a technique, a large quantity of cartilage cells can be obtained without damaging healthy tissue. Further, they discovered that a large quantity of cartilage tissue could be engineered without the use of special scaffolds by conducting culture in a simulated microgravity environment with the use of the Rotating Wall Vessel (RWV) bioreactor. This has led to the completion of the present invention.

Specifically, the present invention concerns a method for engineering cartilage tissue by three-dimensionally cultured bone marrow cells in a simulated microgravity environment.

In this method, the gravity is preferably approximately 1/10 to 1/100 of the ground gravity on a time-average basis in a simulated microgravity environment. Such simulated microgravity environment can be attained with the use of a bioreactor that realizes the simulated microgravity environment on the ground by compensating the ground gravity by the stress resulting from rotation.

A uniaxial rotary bioreactor is preferably used, and an example thereof is the Rotating Wall Vessel (RWV) bioreactor. When the Rotating Wall Vessel (RWV) bioreactor is used, culture is conducted at a seeding density of 10⁶ to 10⁷ cells/cm³ at a rotation speed of approximately 8.5 to 25 rpm (a 5-cm-vessel), for example. It should be noted that the culture conditions are not limited thereto.

In the method of the present invention, it is preferable that an inducer of cartilage differentiation, such as TGF-β or dexamethasone, be added to a culture solution. Further, it is preferable that bone marrow cells be two-dimensionally cultured to confluence, resuspended in the bioreactor, and then subjected to culture in a simulated microgravity environment.

As an embodiment of the present invention, bone marrow cells isolated from a patient are used. Engineered cartilage tissue from bone marrow cells from a patient is free from the risk of rejection or the like to the patient. Thus, such tissue can be preferably used to regenerate and/or repair cartilage defects of a patient.

According to the present invention, three-dimensional cartilage tissue can be effectively engineered in vitro without damaging autologous cartilage.

### BRIEF DESCRIPTION OF THE DRAWINGS

FIG. 1 illustrates the experimentation protocol of Example 1.

FIG. 2 shows photographs of an RWV vessel (upper photograph) and of a 15-ml conical tube (lower photograph).

FIG. 3 shows photographs of stained images of the cartilage tissue sections engineered in Example 1 (top row: hematoxylin-eosin (HE) staining; middle row: alcian blue staining; bottom row: safranin O staining).

FIG. 4 shows a comparison of tissue masses formed after culture (left: rotation culture performed using an RWV with the addition of TGF-β; middle: static culture (pellet culture) performed with the addition of TGF-β; right: static culture (pellet culture) performed without the addition of TGF-β(10% FBS)).

FIG. 5 shows a chart representing a change in the rotation speed of an RWV.

FIG. 6 shows a chart representing the results of the alkaline phosphatase activity assay (left: static culture (pellet culture) performed with the addition of TGF-β; middle: static culture (pellet culture) performed without the addition of TGF-β (10% FBS); right: rotation culture performed using an RWV with the addition of TGF-β).

FIG. 7 shows the results of RT-PCR (A: collagen type II; B: aggrecan) (left chart: static culture (pellet culture) performed with the addition of TGF-β; right chart: rotation culture using an RWV).

FIG. 8 shows a chart representing a comparison of the compressive strength of the cartilage tissue 4 weeks after culture (left) and that of the articular cartilage tissue of a normal rabbit (right).

FIG. 9 shows photographs of macroscopic appearance 4 weeks after the transplantation of cultured tissue (cultured for 2 weeks in vitro) into an osteochondral defect of a rabbit knee joint (A: cartilage tissue cultured using an RWV (bar: 10 mm); B: osteochondral defect (bar: 5 mm); C: observation immediately after transplantation; D: observation 4 weeks after transplantation).

FIG. 10 shows a chart representing a comparison of the hardness of a site of transplantation (left) and that of the articular cartilage tissue of a normal rabbit (right).

FIG. 11 shows photographs of HE-stained images of transplanted tissue (the site of transplantation is surrounded with a frame) (A: rabbit articular cartilage tissue; B: transplanted tissue).

FIG. 12 shows photographs of safranin-O-stained images of transplanted tissue (A: rabbit articular cartilage tissue; B: transplanted tissue).

FIG. 13 shows photographs of immunohistologically stained images of transplanted tissue (A: rabbit articular cartilage tissue; B: transplanted tissue).

This description includes part or all of the contents as disclosed in the descriptions of Japanese Patent Application Nos. 2003-413758 and 2004-96686, which are priority documents of the present application.

### PREFERRED EMBODIMENTS OF THE INVENTION

Hereafter, the present invention is described in detail.

1. Simulated Microgravity Environment

In the present invention, the term “simulated microgravity environment” refers to a simulated microgravity environment that imitates the microgravity environment found in space. Such simulated microgravity environment is realized by compensating the ground gravity by the stress resulting from rotation, for example. More specifically, a rotating substance receives a force that is represented by the vector sum of the ground gravity and the stress, and thus, the magnitude and the direction thereof vary depending on time. That is, excessively smaller force than the ground gravity (g) acts on a substance on a time-average basis. This allows realization of a “simulated microgravity environment” that is very similar to space.

In a “simulated microgravity environment,” it is necessary that cells grow, differentiate, and are homogeneously dispersed without sinking and that they three-dimensionally aggregate to form tissue. In other words, the rotation speed is preferably adjusted to synchronize with the sinking speed of the seeded cells to minimize the influence of the ground gravity on the cells. More specifically, the microgravity applied onto the cultured cells is preferably approximately 1/10 to 1/100 of the ground gravity (g) on a time-average basis.

2. Bioreactor

In the present invention, a rotary bioreactor is used in order to realize a simulated microgravity environment. Examples of bioreactors that can be used include the Rotating-Wall Vessel (RWV, U.S. Pat. No. 5,002,890), the Rotary Cell Culture System™ (RCCS, Synthecon Incorporated), a 3D-clinostat, and bioreactors disclosed in JP Patent Publication (Unexamined) Nos. 8-173143 (1996), 9-37767 (1997), and 2002-45173. RWV and RCCS are particularly preferable because they are equipped with gas exchange membranes. A uniaxial rotary bioreactor is preferable to a biaxial bioreactor for the following reasons. When a biaxial bioreactor (such as a biaxial clinostat) is used, shear stress cannot be minimized, and a sample itself rotates. Thus, a sample cannot be maintained at a stationary position in a vessel, as in a case involving the use of a uniaxial reactor. This stationary state is an important condition for attaining a large three-dimensional tissue mass without the use of specific scaffolds.

The RWV that is used in the examples of the present invention is a NASA-developed uniaxial rotary bioreactor equipped with gas exchange membranes. The RWV bioreactor, which is a horizontal cylindrical bioreactor, is filled with a culture medium, the cells are seeded therein, and the bioreactor rotates along the horizontal axis of the cylinder to conduct culture. The “microgravity environment” that provides a considerably lower gravity than the ground gravity is virtually realized in the bioreactor because of the stress resulting from rotation. In such simulated microgravity environment, cells are homogeneously suspended in a culture medium, they are cultured and grow under the minimal shear stress for a necessary period of time, and they aggregate to form tissue masses.

A preferable rotation speed when an RWV is used is adequately determined in accordance with the diameter of the vessel and the size or mass of the tissue mass. When a 5-cm vessel is used, for example, the rotation speed is preferably between about 8.5 rpm and 25 rpm. When culture is conducted at such rotation speed, the gravity acting on the cells in the vessel is substantially about 1/10 to 1/100 of the ground gravity (1g).

3. Bone Marrow Cells

In the present invention, bone marrow cells are used as cell sources for cartilage tissue engineering. The bone marrow cells used in the present invention are undifferentiated cells that are capable of differentiation and growth. Bone-marrow-derived mesenchymal stem cells are particularly preferable. In addition to the established cell lines, bone marrow cells isolated from patients are preferably used. Such cells are preferably prepared by isolating the bone marrow cells from the patient and removing connective tissue and the like therefrom in accordance with a conventional technique. Alternatively, primary culture may be conducted in accordance with a conventional technique and cells may grow in advance. Further, the cultured cells isolated from the patient may be cryopreserved. Specifically, the bone marrow cells that have been isolated in advance may be cryopreserved and then used according to need.

4. Cell Culture Conditions

Culture media that are usually employed for culture of bone marrow cells, such as MEM, α-MEM, and DMEM, can be adequately selected in accordance with the type of cells and used for cell differentiation and multiplication. Such media may additionally contain, for example, FBS (Sigma) or antibiotics such as Antibiotic-Antimycotic (Gibco BRL).

A culture media may further contain at least one member selected from the group consisting of immunosuppressants such as dexamethasone, FK-506, or ciclosporin, bone morphogenetic proteins (BMP) such as BMP-2, BMP-4, BMP-5, BMP-6, BMP-7, or BMP-9, and osteogenic humoral factors such as TGF-β capable of accelerating cartilage cell differentiation in combination with a phosphagen such as glycerol phosphate or ascorbic acid phosphate. Addition of either or both TGF-β and dexamethasone in combination with an adequate phosphagen is particularly preferable. In such a case, the amount of TGF-β added is approximately between 1 ng/ml and 10 ng/ml, and that of dexamethasone added is up to a maximum of 100 nM.

Cell culture is preferably conducted in the presence of 3% to 10% CO₂ at 30° C. to 40° C., and particularly preferably in the presence of 5% CO₂ at 37° C. The duration of culture is not particularly limited, and it is at least for 7 days, and preferably between 21 and 28 days.

When an RWV (a 5-cm vessel) is used, bone marrow cells are seeded at a density of 10⁶ to 10⁷ cells/cm³, and culture is conducted at a rotation speed of 8.5 to 25 rpm (a 5-cm vessel). Under such conditions, the sinking speed of the seeded cells synchronizes with the rotation speed of the vessel, and the influence of the ground gravity imposed on the cells is minimized. When the cells that have been two-dimensionally cultured to overconfluence are subcultured and then cultured using an RWV, a large tissue mass can be obtained.

5. Applications of the invention

The application of the method according to the present invention to regenerative medicine enables cartilage tissue regeneration with the use of autologous bone marrow cells. Specifically, bone marrow cells isolated from a patient are three-dimensionally cultured in a simulated microgravity environment to engineer cartilage tissue, and the engineered cartilage tissue is applied to a cartilage defect of a patient. The engineered cartilage tissue is free from the risk of rejection, and the level of damage imposed on normal tissue resulting from the use of the engineered cartilage tissue is lower than that resulting from the use of autologous cartilage. Thus, use of such engineered cartilage tissue enables safer cartilage regeneration.

### EXAMPLES

### Example 1

### Carrtilage Tissue Engineering from Mesenchymal Stem Cells Derived from Rabbit Bone Marrow

1. Culture of Mesenchymal Stem Cells Derived from Rabbit Bone Marrow

(1) Preparation of Mesenchymal Stem Cells Derived from Rabbit Bone Marrow

Mesenchymal stem cells derived from rabbit bone marrow were extracted from the femur of a 2-week-old JW rabbit (female) in accordance with the method of Maniatopoulos et al. (Maniatopoulos, C., Sodek, J., and Melcher, A. H., 1988, Cell Tissue Res., 254, pp. 317-330). The sampled cells were cultured in DMEM containing 10% FBS (Sigma) and Antibiotic-Antimycotic (GIBCO BRL) for 3 weeks, and they were allowed to grow.

(2) Culture of Mesenchymal Stem Cells Derived from Rabbit Bone Marrow

The mesenchymal stem cells derived from rabbit bone marrow thus prepared were suspended in 10 ml of DMEM culture medium(Sigma) containing 10⁻⁷ M dexamethasone (Sigma), 10 ng/ml TGF-β3 (Sigma), 50 μ g/ml ascorbic acid (Wako), ITS +Premix (BD), 40 μg/ml L-proline (Sigma), and Antibiotic-Antimycotic (GIBCO BRL) to a cell concentration of 1×10⁶ cells/ml, and the resultant was subjected to static culture (pellet culture) or rotation culture using an RWV bioreactor (Synthecon) for 4 weeks.

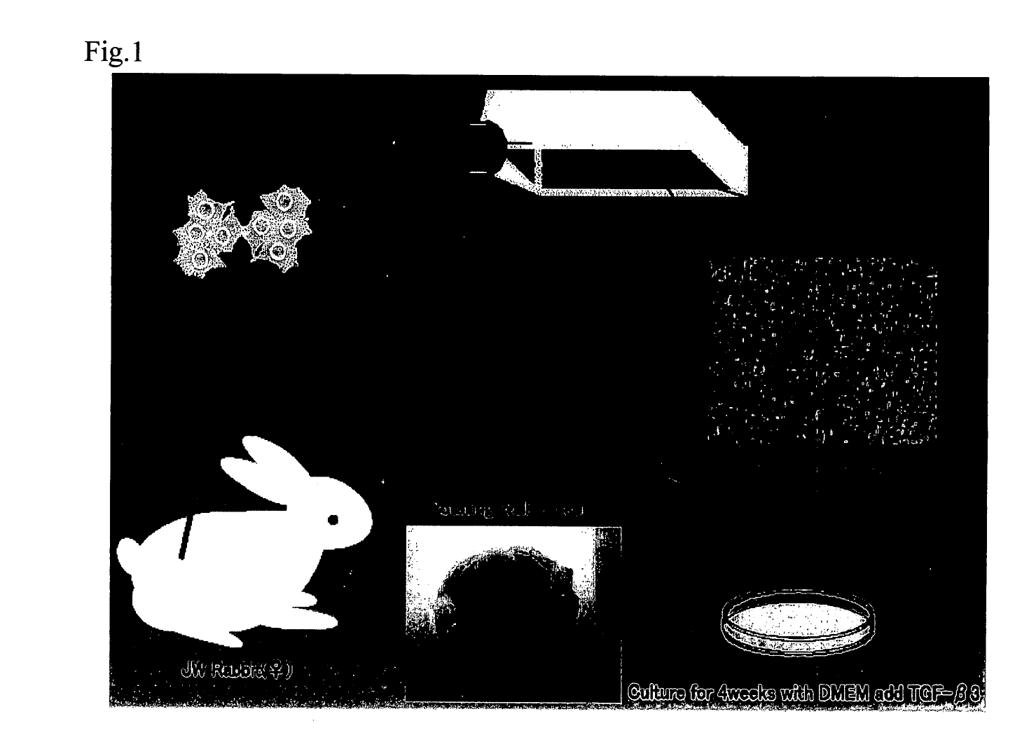

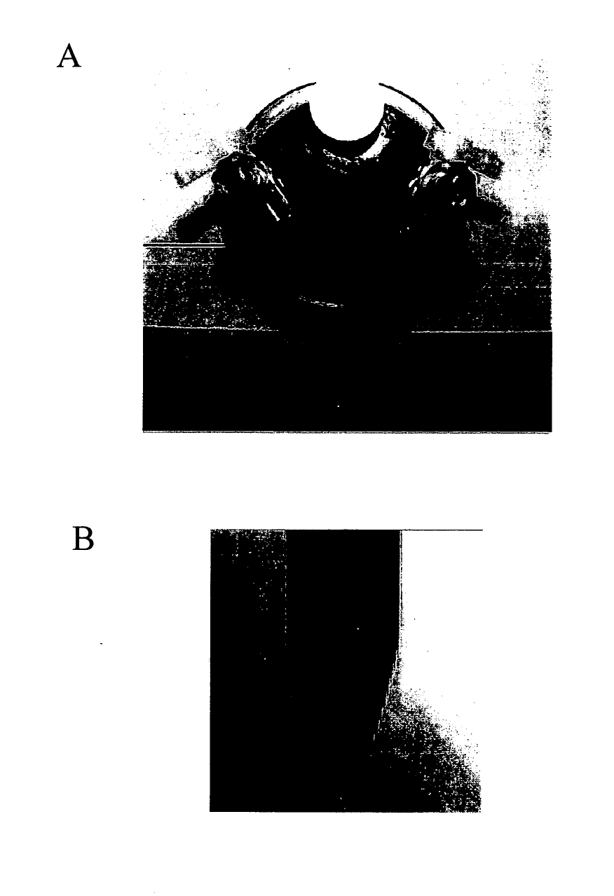

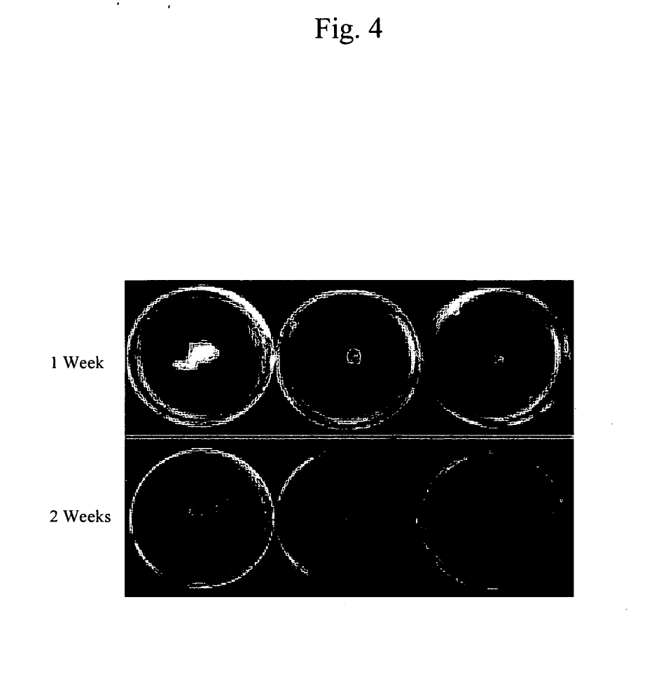

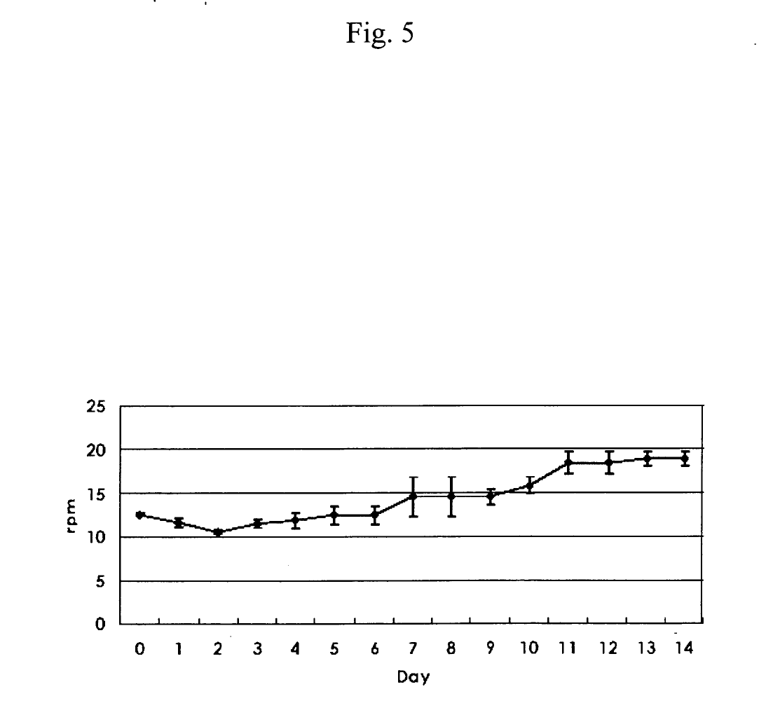

Static culture was conducted by introducing 10 ml of the cell suspension into a 15-ml conical tube, subjecting the tube to centrifugation at 50 g for 5 minutes to prepare the tissue pellet, and subjecting the tissue pellet to culture at 37° C. in the presence of 5% CO₂. Pellet culture was also performed in the same manner, except that TGF-β was not added. Rotation culture using an RWV bioreactor was carried out using a 5-cm vessel at a rotation speed of 8.0 to 24 rpm at 37° C. in the presence of 5% CO₂. The rotation speed was frequently adjusted manually by visually inspecting the cell aggregate to maintain a stationary position in a vessel (time course of the rotation speed of an RWV is shown in FIG. 5). Bubbles would occur because of cellular respiration, and it would disturb the simulated microgravity environment. Thus, bubbles were frequently removed. FIG. 1 shows the protocol of the present example, and FIG. 2 shows photographs of an RWV vessel and of a 15-ml conical tube. FIG. 4 shows the results of a comparison of tissue masses after culture, showing, from left to right, the result of rotation culture performed using an RWV with the addition of TGF-β, that of static culture (pellet culture) performed with the addition of TGF-β, and that of static culture (pellet culture) performed without the addition of TGF-β.

2. Evaluation of Cultured Tissue

(1) Histological Staining

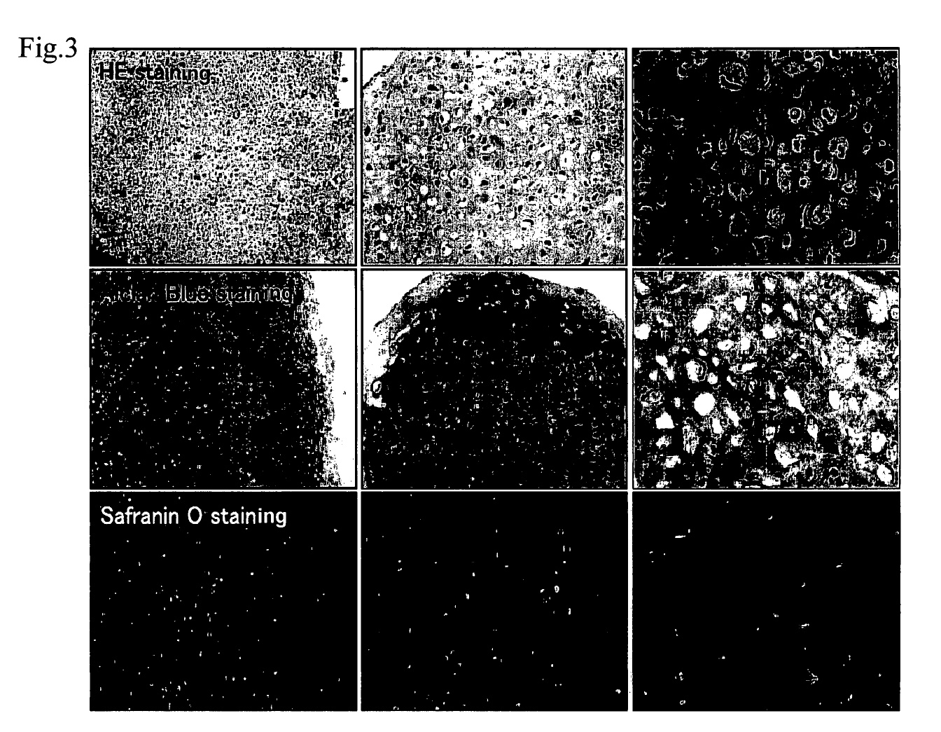

The cartilage tissue obtained by static culture (pellet culture) and that obtained by rotation culture were histologically stained with hematoxylin-eosin (HE), safranin 0, and alcian blue each week to evaluate capacity for cartilaginous matrix formation. The cultured tissues were fixed with 4% paraformaldehyde and 0.1% glutaraldehyde by microwave radiation. On the next day, the resultants were subjected to decalcification in 10% EDTA and 100 mM Tris (pH 7.4), and decalcification was continued for about 1 week. After the decalcification, the resultants were dehydrated in ethanol and then embedded in paraffin. Sections with a thickness of 5 μm each were prepared. Those sections were then deparaffinized and stained with hematoxylin-eosin, safranin O , and alcian blue in accordance with a conventional technique. The results are shown in FIG. 3.

(2) Alkaline Phosphatase Activity

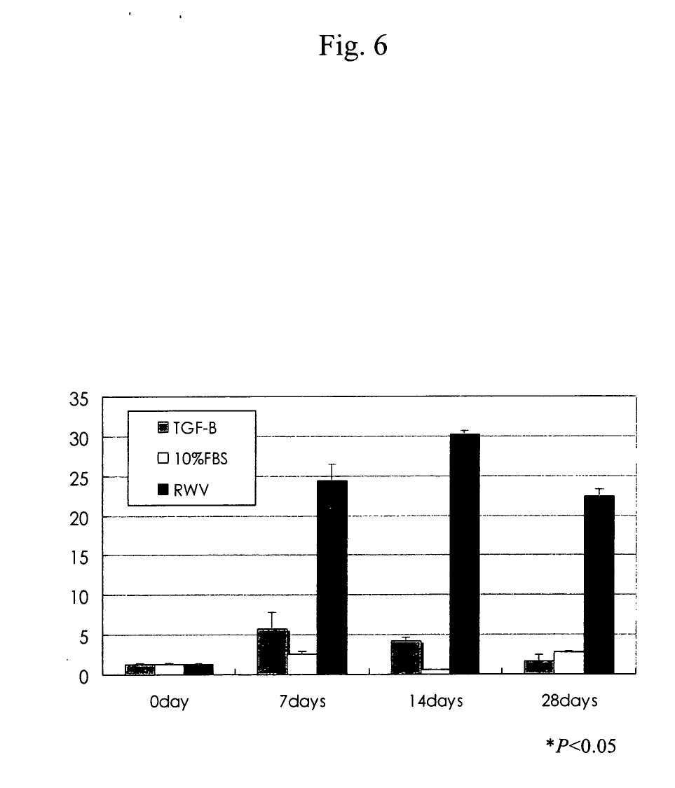

The cartilage tissues obtained by static culture (pellet culture) and by rotation culture were subjected each week to measure the alkaline phosphatase (ALP) activity. ALP activity was measured in the following manner. The cultured tissue was washed with 100 mM Tris (pH 7.5) and 5 mM MgCI₂, colleted using a scraper, suspended in 500 μl of 100 mM Tris (pH 7.5), 5 mM MgCl₂, and 1% Triton X-100, and then disrupted by sonication. After the sonication, cells were centrifuged at 6,000 g for 5 minutes to recover the supernatant. Enzyme activity was determined in the following manner. That is, the supernatant (5 μl in each case) was added to 0.056 M 2-amino-2-methyl-1,3-propanediol (pH 9.9), 10 mM p-nitrophenyl phosphate, and 2 mM MgCI₂, the resultants were incubated at 37° C. for 30 minutes, and absorbance at 405 nm was measured using a microplate reader immediately thereafter. The calibration curve was prepared using p-nitrophenol. The results are shown in FIG. 6. In the chart, “RWV” represents the result of rotation culture using an RWV, “TGF-0“represents the result of pellet culture performed with the addition of TGF-β, and “10% FBS” indicates the result of pellet culture performed without the addition of TGF-β.

(3) Quantitative RT-PCR

The cartilage tissue obtained by static culture (pellet culture) and that obtained by rotation culture were subjected each week to quantitative RT-PCR to examine the expression levels of cartilage-specific genes, such as collagen type II and aggrecan.

Total RNA was extracted from the cultured tissue using a TRIzol reagent (Invitrogen) in accordance with the protocol. The cultured tissue was lysed in TRIzol, 200 μl of chloroform was added thereto, they were thoroughly mixed by shaking, and the resulting mixture was centrifuged at 15,000 rpm. After isopropanol precipitation and ethanol precipitation, the resultant was dissolved in DEPC water, the density was assayed based on the results of absorbance assay, and approximately 1 fg of total RNA was subjected to RT-PCR.

RT-PCR was carried out using the First-Strand cDNA Synthesis Using SuperScript III for RT-PCR kit (Invitrogen) and the TaKaRa RNA PCR kit (AMV) Ver. 2.1 (TaKaRa). With the use of the First-Strand cDNA Synthesis Using SuperScript III for RT-PCR, RT-PCR was carried out at 50° C. for 60 minutes and then at 70° C. for 15 minutes. With the use of the TaKaRa RNA PCR kit (AMV) Ver. 2.1 (TaKaRa), RT-PCR was carried out at 30° C. for 10 minutes, at 42° C. for 30 minutes, at 99° C. for 5 minutes, and at 5oC for 5 minutes. The primers used for RT-PCR are as shown below.

[RT-primers]
Aggrecan:

(SEQ ID NO:1)

5′-cctaccaggacaaggtctcg-3′
Collagen type II:

(SEQ ID NO:2)

5′-ccatcattgacattgcacccatgg-3′

Real-time PCR was carried out using the FastStart DNA Master CYBR Green 1 kit, the LightCycler PCR apparatus (Roche), and the following primers under the following reaction conditions.

[PCR primers]
Aggrecan Forward:

(SEQ ID NO:3)

5′-cctaccaggacaaggtctcg-3′
Aggrecan Reverse:

(SEQ ID NO:4)

5′-gtagcctcgctgtcctcaag-3′
Collagen type II Forward:

(SEQ ID NO:5)

5′-ccatcattgacattgcacccatgg-3′
Collagen type II Reverse:

(SEQ ID NO:6)

5′-gttagtttcctgtctctgccttg-3′

[PCR Conditions]
Denaturation: One Cycle of 95° C. for 5 seconds
Amplification: 40 Cycles of 95° C. for 15 seconds, 60° C. for 5 seconds, and 72° C. for 15seconds
Melting Curve: 70° C. for 10 Seconds Cooling: 40° C. for 30 seconds

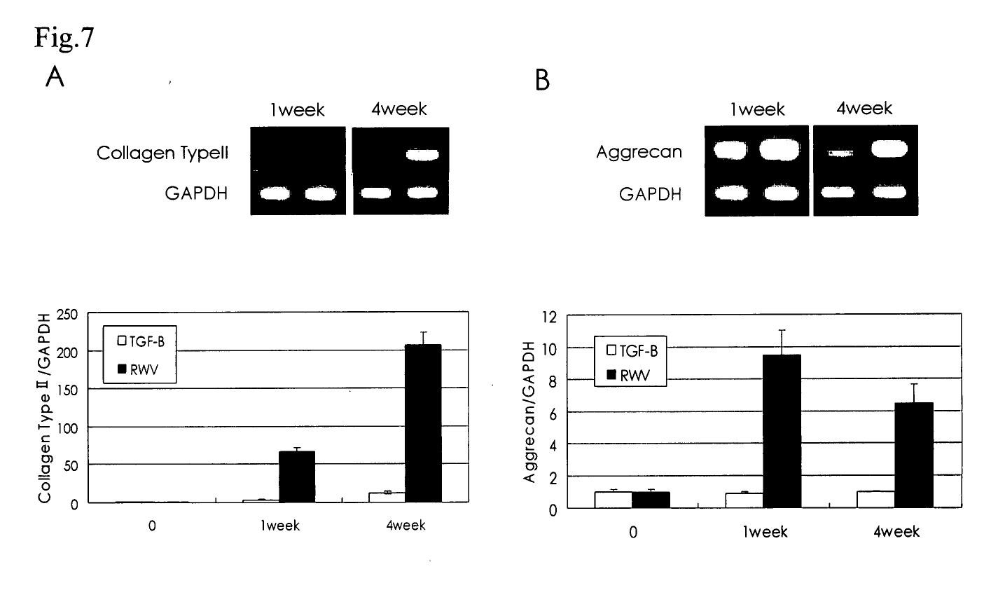

FIG. 7 shows the results of RT-PCR (A: collagen type II; B: aggrecan). In the chart, “RWV” represents the results of rotation culture performed with the use of an RWV and “TFG-β” represents the results of pellet culture performed with the addition of TGF-β.
3. Results

Three weeks later, cells sank onto the bottom, cell aggregation was weak, and the tissue diameter was approximately 5 mm as a result of static culture (pellet culture). In contrast, cells had become aggregated with each other in a simulated microgravity environment, and three-dimensional tissue with a diameter of approximately 1 cm to 1.5 cm was generated as a result of rotation culture using an RWV bioreactor. This three-dimensional tissue was stained with safranin 0 and alcian blue, which indicates that this tissue has the capacity for cartilaginous matrix formation. Based on the results of quantitative RT-PCR, collagen type II and aggrecan expression was observed. These results indicate that three-dimensional cartilage tissue can be regenerated from bone-marrow-derived mesenchymal stem cells with the use of the RWV bioreactor.

Further, the optimal culture conditions with the use of the RWV were examined. This revealed that a large tissue mass could be obtained by two-dimensionally culturing cells to overconfluence, subjecting the resulting cells to subculture, and then culturing them with the use of the RWV.

### Example 2

### Assay of Strength of RWV-Xultured Tissue

Mechanical strength of the RWV-cultured tissue was measured using the EIKO TA-XT2i (Eko Instruments). The RWV-cultured tissue prepared by the procedure of Example 1 was cut into 2-mm square pieces and then compressed at a rate of 0.1 mm/sec. The stress-strain curve was determined from the compression load (Pa) and the distance (mm), and the strength was calculated based thereon.

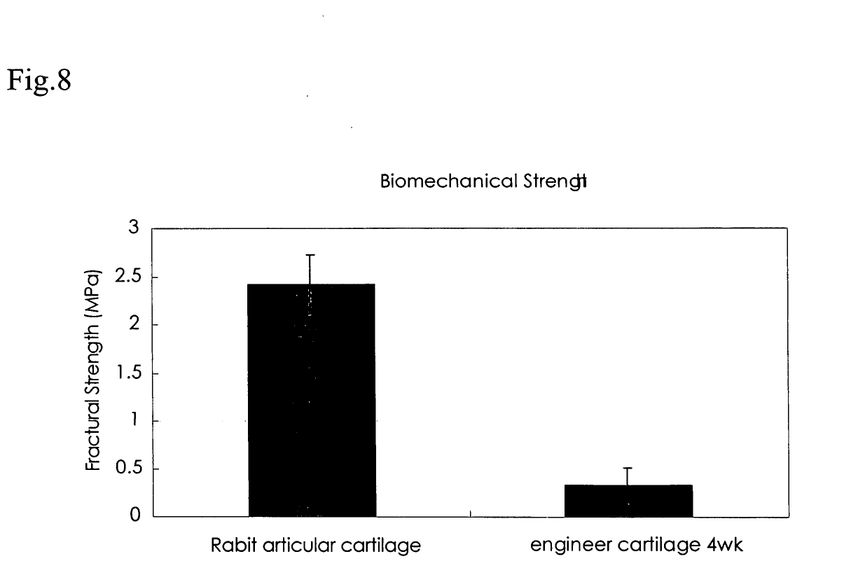

FIG. 8 shows the results of a comparison of the compression strength of the cartilage tissue 4 weeks after the initiation of culture and that of the articular cartilage tissue of a rabbit.

### Example 3

### Experimentation Concerning Transplantation of RWV-Cultured Tissue Into Osteochondral Defect in Rabbit Knee Joint

1. Transplantation into Osteochondral Defect in Rabbit Knee Joint

The RWV-cultured tissue prepared by the procedure of Example 1 (cultured in vitro for 2 weeks) was transplanted into an osteochondral defect in a rabbit knee joint, and the hardness at the site of transplantation and the results of histological observation thereof were evaluated.

The rabbit was intravenously anesthetized with 0.6 mg/kg of Somnopentyl. The weight-bearing area of the left femoral condyle (the left knee joint) was designated as the site of surgical operation. A vertical skin incision was made on the lateral side of the patella, and the articular capsule was incised through a medial parapatellar approach. The patella was dislocated via lateral reflection, and an osteochondral defect 4 mm in depth was provided in the femoral trochlea using a drill with a diameter of 5 mm (the bottom surface was smoothened using a flat-ended drill, and the periphery was trimmed using a scalpel). A cartilage mass was cut into 5-mm pieces using a leather hole puncher, and the resulting pieces were transplanted into the defect. The patella was repositioned, the articular capsule and the skin were sutured with 4-0 nylon, the flexion and extension of the knee joint were observed to confirm that the patella would not be dislocated, and the surgical operation was then completed.

2. Hardness of Transplanted Tissue

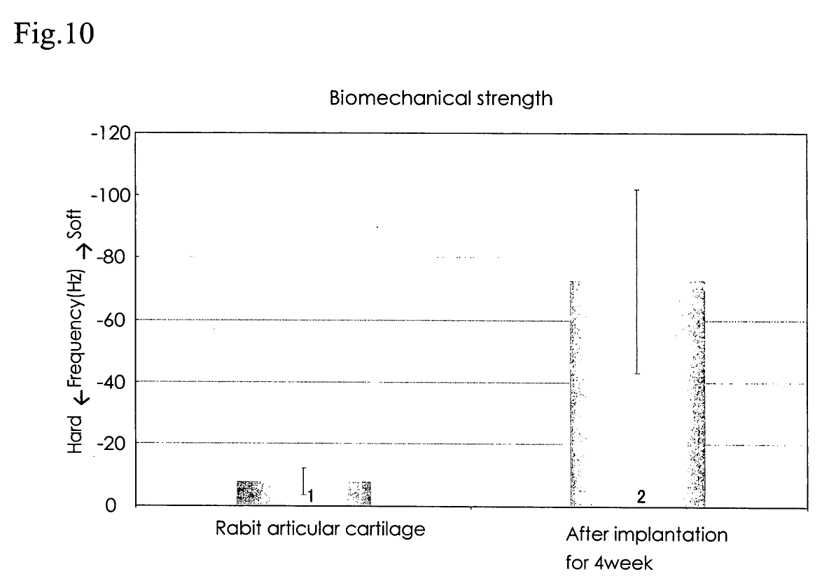

The hardness of the transplanted tissue was measured by applying a probe to the site of measurement and detecting a change in frequency using the Venus Rod (Axiom). FIG. 10 shows the result of measuring the hardness at the site of transplantation (left) and that at the articular cartilage tissue of a normal rabbit (right).

3. Histological Observation

The transplanted tissue was evaluated by hematoxylin-eosin (HE) staining, safranin 0 (SO) staining, and immunohistological staining, in addition to macroscopic appearance observation.

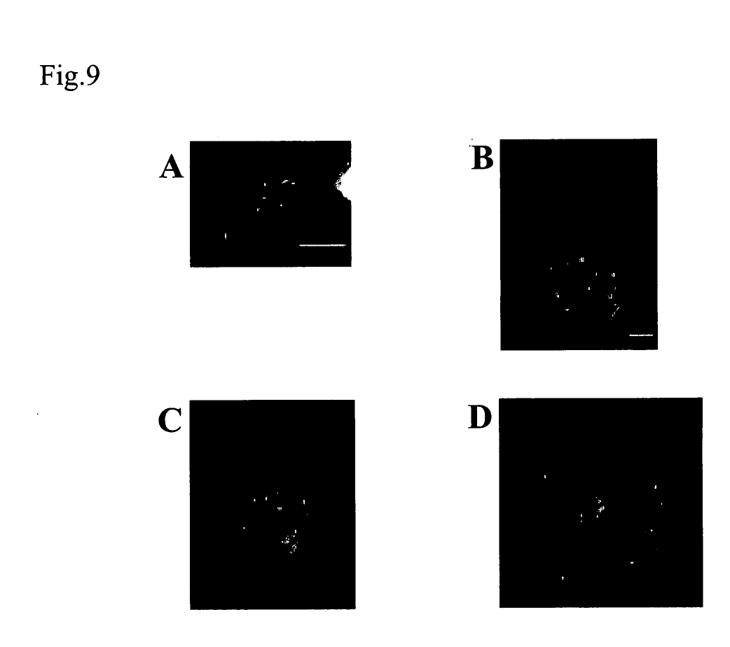

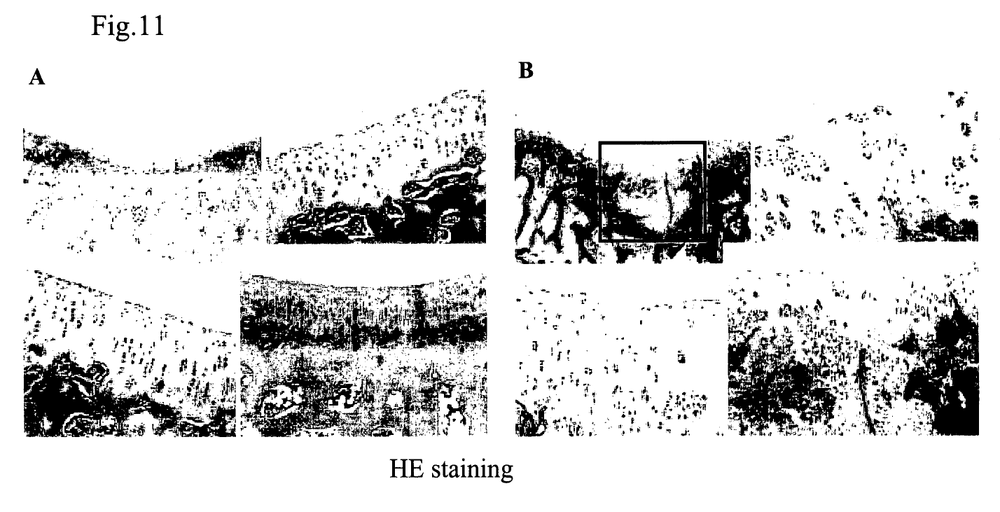

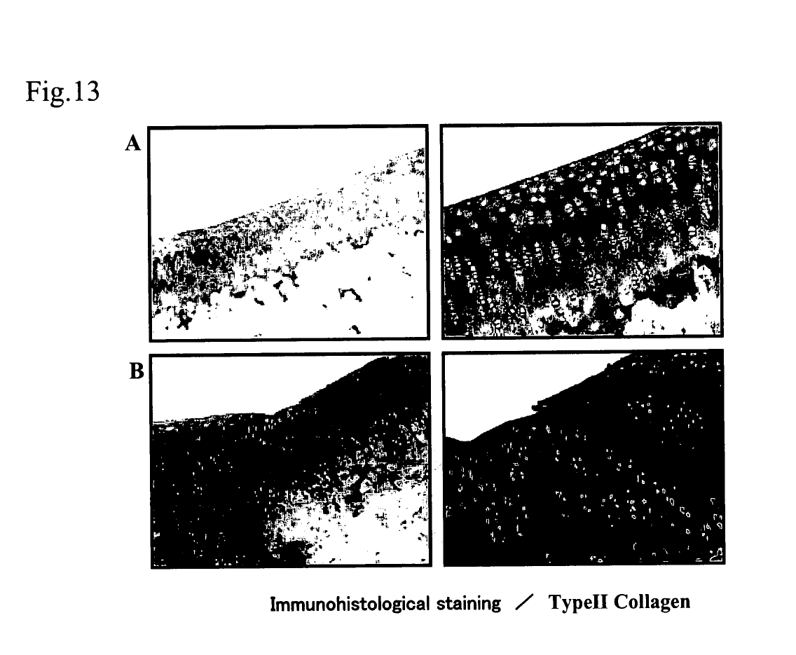

FIG. 9 shows photographs showing the RWV-cultured tissue 4 weeks after the transplantation (A: cartilage tissue cultured using an RWV (bar: 10 mm); B: osteochondral defect (bar: 5 mm); C: observation immediately after transplantation; D: observation 4 weeks after transplantation). FIG. 11 to FIG. 13 each show the result of HE staining, that of SO staining, and that of immunohistological staining of the transplanted tissue (A: rabbit articular cartilage tissue; B: transplanted tissue).

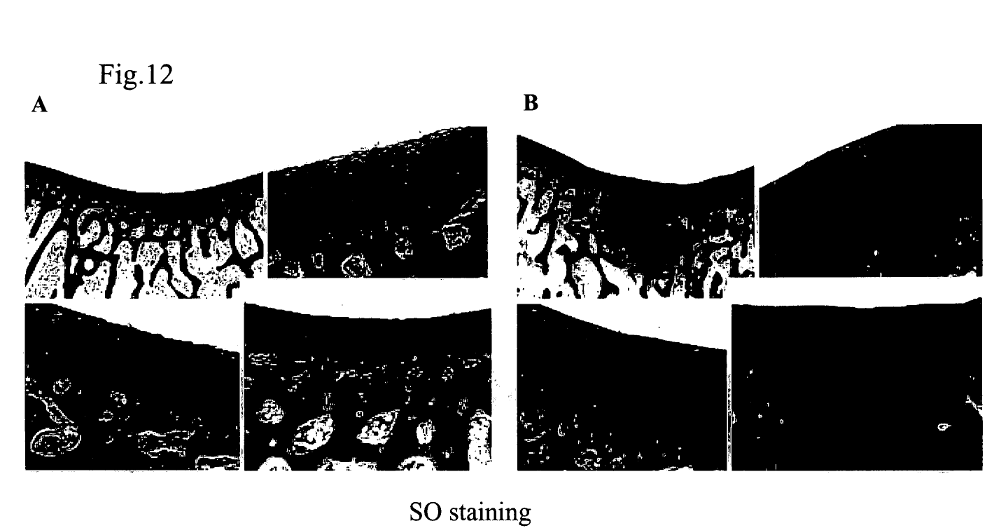

As a result of rotation culture using an RWV for 4 weeks, cartilage tissue with a greater diameter of 15 mm was engineered (FIG. 9(A)). As a result of the histological observation of the defects of an osteochondral defect model 4 weeks after the transplantation (FIG. 9(B) (C)), a very smooth surface was observed. This indicates that cartilage regeneration was satisfactorily achieved. Based on observation of the HE-stained image of the tissue sections 4 weeks after the transplantation, the regenerated cartilage was found to be as satisfactory as the normal cartilage tissue (FIG. 11). Based on observation of the safranin O-stained image, in which safranin O specifically stained the cartilaginous matrix, the stained image was found to be similar to that of normal cartilage tissue. Thus, it was confirmed that cartilage was regenerated while producing a cartilaginous matrix (FIG. 12). Expression of cartilage-specific type II collagen was also observed.

All publications, patents, and patent applications cited herein are incorporated herein by reference in their entirety.

Industrial Applicability

According to the present invention, cartilage tissue can be effectively engineered from bone marrow cells without damaging autologous cartilage. The method of the present invention can be applied to regenerative medicine aimed at repairing cartilage defects as well as to basic research.

Sequence Listing Free Text

SEQ ID NO: 1: description of artificial sequence: synthetic DNA (primer)
SEQ ID NO: 2: description of artificial sequence: synthetic DNA (primer)
SEQ ID NO: 3: description of artificial sequence: synthetic DNA (primer)
SEQ ID NO: 4: description of artificial sequence: synthetic DNA (primer)
SEQ ID NO: 5: description of artificial sequence: synthetic DNA (primer)
SEQ ID NO: 6: description of artificial sequence: synthetic DNA (primer)

1. A method for engineering cartilage tissue by three-dimensionally culturing bone marrow cells in a simulated microgravity environment.

2. The method according to claim 1, wherein the simulated microgravity environment provides gravity that is 1/10 to 1/100 of the ground gravity to an object on a time-average basis.

3. The method according to claim 1, wherein the simulated microgravity environment is attained with the use of a bioreactor that realizes a simulated microgravity environment on the earth by compensating the ground gravity with the stress resulting from rotation.

4. The method according to claim 3, wherein the bioreactor that realizes a simulated microgravity environment on the ground is a uniaxial rotary bioreactor.

5. The method according to claim 4, wherein the bioreactor that realizes a simulated microgravity environment on the ground is a Rotating Wall Vessel (RWV) bioreactor.

6. The method according to claim 5, wherein culture is conducted by seeding bone marrow cells at a density of 10⁶ to 10⁷ cells/cm³ at a rotation speed of 8.5 to 25 rpm when a 5-cm RWV vessel is used.

7. The method according to claim 1, wherein culture is conducted by adding TGF-βand/or dexamethasone to a culture medium.

8. The method according to claim 1, wherein bone marrow cells are two-dimensionally cultured to confluence, subcultured, and then cultured in a simulated microgravity environment.

9. The method according to claim 1, wherein the bone marrow cells are isolated from a subject in need of transplantation of the engineered cartilage tissue.
